SEQUENCE LISTING (4 sequences)

SEQ ID NO: 1 - DNA, 3822 bp, Human coronavirus
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 2 - protein, 1273 aa, Human coronavirus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - DNA, 3822 bp, Artificial Sequence
  Codon-optimized human coronavirus
atgtttgtctttcttgtgcttcttcctttggtcagttcacagtgtgtcaatcttactaccaggacacaacttccaccggcatacactaattctttcacacggggagtctattaccctgacaaagtgttcagaagttctgtgctgcattcaacacaagatcttttcctcccgttctttagtaatgtgacctggtttcatgcaattcatgtgtcaggaaccaatggtacaaagcggttcgataaccctgtgttgccgttcaatgacggagtctactttgcttcaactgagaaaagtaacatcattcggggatggattttcggaactacactcgattctaagacccagagtcttttgattgtgaataacgcaacaaatgtcgtcatcaaagtctgtgagtttcaattttgtaatgatccatttcttggagtgtattaccacaaaaacaataagtcatggatggagtcggagtttcgggtctattcttccgctaataattgcacttttgagtacgtctcacagccatttcttatggaccttgagggaaagcagggaaatttcaaaaatcttagggagtttgtgtttaagaacattgacggatacttcaaaatctattcaaagcacactccgatcaacctggtcagggatcttccgcaaggtttttcagcacttgaacctcttgtcgatctgcctattgggatcaacatcacaagattccaaactttgcttgctttgcatcggtcctatctcactccgggagattctagttcagggtggacagctggagcagctgcttactatgtgggttatcttcaacctaggacttttctgctgaaatacaatgagaatggaaccatcactgacgctgtcgattgtgcactcgaccctctgtcagaaaccaaatgtactctcaaatctttcactgtggagaagggaatctaccaaacttcaaacttcagagtgcagcctacagagtctattgtgcggtttccgaatatcactaacctttgcccgtttggagaagtgttcaatgccactagatttgcttcagtctatgcttggaaccggaaacggattagcaactgtgtcgctgactattcagtgctttacaattcagcatctttctcaacctttaagtgctacggagtgtctccgacaaaactcaatgacctttgtttcactaatgtctatgctgactcgttcgtcattcggggagatgaagtccggcagattgccccagggcaaacaggcaaaatcgctgactataactacaaactgcctgacgactttaccggatgcgtcattgcatggaatagtaacaatctcgattctaaagtcggtggaaactacaattacctctatagactttttcggaagtcaaatctcaaaccttttgaacgggacatctctactgagatctaccaagctggtagtacaccatgtaatggagtggagggattcaattgctacttccctttgcagtcctacggtttccaacccacaaatggagtcggttaccagccttatagggtcgtcgtcttgtcgtttgagttgcttcatgcacctgctacagtctgtggtccaaagaaatctactaatcttgtcaaaaacaaatgtgtgaatttcaacttcaatggacttactggtactggtgtccttactgaatctaacaaaaagtttctgccttttcaacaatttggtagagatattgctgatactactgatgctgtccgggacccacagactcttgaaattctcgacattactccatgttcgtttggaggtgtctcagtcattacacctggtactaatactagcaaccaggtcgctgtcttgtatcaggatgtgaactgcacagaagtgccagtggctatccatgcagatcaattgactccaacttggcgggtctattcgactggatcaaatgtgtttcaaactagggccggatgtttgattggagctgagcatgtcaataactcttatgagtgtgacattccgattggagcaggaatctgcgcttcttatcaaactcaaacaaattcaccaagaagagcacggagcgtcgcttcacaatctatcatcgcatacacaatgtcacttggagctgagaatagtgtcgcttactctaacaattcaattgccattcctactaattttacaatctcagtcaccaccgaaatccttccagtgtcaatgaccaagacttcagtcgattgtacaatgtacatctgtggtgattctaccgagtgtagtaatcttctccttcaatatgggtcattttgtacacagctcaatagagctttgactggaatcgcagtggagcaagacaaaaacactcaagaggtctttgcacaagtcaagcaaatctacaaaacaccaccgatcaaagattttggaggattcaatttctcacaaattcttccggacccgtcaaaacctagcaagagatctttcatcgaagatttgcttttcaataaggtgacacttgctgatgctggtttcatcaaacaatatggcgattgtcttggtgatatcgcagctcgggacctgatctgtgcacaaaagtttaacggactgactgtcctccccccacttctgactgatgagatgattgctcaatacacaagtgctctgcttgctggaactatcacatcgggatggacctttggagctggagctgcacttcaaattccttttgctatgcaaatggcttatcggtttaacggtattggtgtcacacagaatgtcctttatgaaaatcaaaagcttatcgccaaccaattcaatagtgctattgggaagattcaagacagtctttcatctactgcttctgctctcggtaaacttcaagatgtcgtcaatcaaaatgctcaagcattgaacactcttgtcaagcagcttagtagtaactttggagctatttcttctgtgcttaatgacatcctttcacggcttgacaaggtcgaagcagaggtgcaaattgataggcttatcactgggagacttcaaagtcttcagacctatgtcactcaacaacttattcgggctgctgaaatccgggcatcagccaatttggcagccactaagatgtcagagtgtgtcttgggacaatctaaaagagtcgatttctgtggtaaggggtatcatcttatgtcattcccgcagtcagctccgcatggagtcgtctttttgcatgtgacttatgtcccagcacaagaaaagaacttcactactgcaccagctatttgtcatgatggaaaagctcactttccacgggaaggagtgtttgtgtctaatggaactcattggttcgtcacacagaggaatttctatgaaccgcagattatcactacagacaacacttttgtcagtggtaactgcgatgtcgtcattgggattgtcaacaacaccgtgtacgatccgcttcaaccggaactcgattctttcaaggaggaacttgacaaatatttcaagaatcatacatcacctgatgtggatcttggagacatctcgggaatcaatgcatcggtcgtcaatatccaaaaggaaattgaccggttgaatgaggtggccaagaatttgaatgagtcacttattgatctccaagagttgggaaagtatgaacagtatatcaaatggccatggtacatttggcttggattcatcgctggcctgattgccatcgtcatggtgaccattatgctctgttgtatgacctcatgctgttcttgtctcaagggatgttgttcatgtgggtcgtgctgcaaatttgatgaggacgactcagaaccagtgctcaaaggagtcaaactccattacacttga

SEQ ID NO: 4 - RNA, 3822 nt, Artificial Sequence
  codon-optimized human coronavirus
auguuugucuuucuugugcuucuuccuuuggucaguucacagugugucaaucuuacuaccaggacacaacuuccaccggcauacacuaauucuuucacacggggagucuauuacccugacaaaguguucagaaguucugugcugcauucaacacaagaucuuuuccucccguucuuuaguaaugugaccugguuucaugcaauucaugugucaggaaccaaugguacaaagcgguucgauaacccuguguugccguucaaugacggagucuacuuugcuucaacugagaaaaguaacaucauucggggauggauuuucggaacuacacucgauucuaagacccagagucuuuugauugugaauaacgcaacaaaugucgucaucaaagucugugaguuucaauuuuguaaugauccauuucuuggaguguauuaccacaaaaacaauaagucauggauggagucggaguuucgggucuauucuuccgcuaauaauugcacuuuugaguacgucucacagccauuucuuauggaccuugagggaaagcagggaaauuucaaaaaucuuagggaguuuguguuuaagaacauugacggauacuucaaaaucuauucaaagcacacuccgaucaaccuggucagggaucuuccgcaagguuuuucagcacuugaaccucuugucgaucugccuauugggaucaacaucacaagauuccaaacuuugcuugcuuugcaucgguccuaucucacuccgggagauucuaguucaggguggacagcuggagcagcugcuuacuauguggguuaucuucaaccuaggacuuuucugcugaaauacaaugagaauggaaccaucacugacgcugucgauugugcacucgacccucugucagaaaccaaauguacucucaaaucuuucacuguggagaagggaaucuaccaaacuucaaacuucagagugcagccuacagagucuauugugcgguuuccgaauaucacuaaccuuugcccguuuggagaaguguucaaugccacuagauuugcuucagucuaugcuuggaaccggaaacggauuagcaacugugucgcugacuauucagugcuuuacaauucagcaucuuucucaaccuuuaagugcuacggagugucuccgacaaaacucaaugaccuuuguuucacuaaugucuaugcugacucguucgucauucggggagaugaaguccggcagauugccccagggcaaacaggcaaaaucgcugacuauaacuacaaacugccugacgacuuuaccggaugcgucauugcauggaauaguaacaaucucgauucuaaagucgguggaaacuacaauuaccucuauagacuuuuucggaagucaaaucucaaaccuuuugaacgggacaucucuacugagaucuaccaagcugguaguacaccauguaauggaguggagggauucaauugcuacuucccuuugcaguccuacgguuuccaacccacaaauggagucgguuaccagccuuauagggucgucgucuugucguuugaguugcuucaugcaccugcuacagucugugguccaaagaaaucuacuaaucuugucaaaaacaaaugugugaauuucaacuucaauggacuuacugguacugguguccuuacugaaucuaacaaaaaguuucugccuuuucaacaauuugguagagauauugcugauacuacugaugcuguccgggacccacagacucuugaaauucucgacauuacuccauguucguuuggaggugucucagucauuacaccugguacuaauacuagcaaccaggucgcugucuuguaucaggaugugaacugcacagaagugccaguggcuauccaugcagaucaauugacuccaacuuggcgggucuauucgacuggaucaaauguguuucaaacuagggccggauguuugauuggagcugagcaugucaauaacucuuaugagugugacauuccgauuggagcaggaaucugcgcuucuuaucaaacucaaacaaauucaccaagaagagcacggagcgucgcuucacaaucuaucaucgcauacacaaugucacuuggagcugagaauagugucgcuuacucuaacaauucaauugccauuccuacuaauuuuacaaucucagucaccaccgaaauccuuccagugucaaugaccaagacuucagucgauuguacaauguacaucuguggugauucuaccgaguguaguaaucuucuccuucaauaugggucauuuuguacacagcucaauagagcuuugacuggaaucgcaguggagcaagacaaaaacacucaagaggucuuugcacaagucaagcaaaucuacaaaacaccaccgaucaaagauuuuggaggauucaauuucucacaaauucuuccggacccgucaaaaccuagcaagagaucuuucaucgaagauuugcuuuucaauaaggugacacuugcugaugcugguuucaucaaacaauauggcgauugucuuggugauaucgcagcucgggaccugaucugugcacaaaaguuuaacggacugacuguccuccccccacuucugacugaugagaugauugcucaauacacaagugcucugcuugcuggaacuaucacaucgggauggaccuuuggagcuggagcugcacuucaaauuccuuuugcuaugcaaauggcuuaucgguuuaacgguauuggugucacacagaauguccuuuaugaaaaucaaaagcuuaucgccaaccaauucaauagugcuauugggaagauucaagacagucuuucaucuacugcuucugcucucgguaaacuucaagaugucgucaaucaaaaugcucaagcauugaacacucuugucaagcagcuuaguaguaacuuuggagcuauuucuucugugcuuaaugacauccuuucacggcuugacaaggucgaagcagaggugcaaauugauaggcuuaucacugggagacuucaaagucuucagaccuaugucacucaacaacuuauucgggcugcugaaauccgggcaucagccaauuuggcagccacuaagaugucagagugugucuugggacaaucuaaaagagucgauuucugugguaagggguaucaucuuaugucauucccgcagucagcuccgcauggagucgucuuuuugcaugugacuuaugucccagcacaagaaaagaacuucacuacugcaccagcuauuugucaugauggaaaagcucacuuuccacgggaaggaguguuugugucuaauggaacucauugguucgucacacagaggaauuucuaugaaccgcagauuaucacuacagacaacacuuuugucagugguaacugcgaugucgucauugggauugucaacaacaccguguacgauccgcuucaaccggaacucgauucuuucaaggaggaacuugacaaauauuucaagaaucauacaucaccugauguggaucuuggagacaucucgggaaucaaugcaucggucgucaauauccaaaaggaaauugaccgguugaaugagguggccaagaauuugaaugagucacuuauugaucuccaagaguugggaaaguaugaacaguauaucaaauggccaugguacauuuggcuuggauucaucgcuggccugauugccaucgucauggugaccauuaugcucuguuguaugaccucaugcuguucuugucucaagggauguuguucaugugggucgugcugcaaauuugaugaggacgacucagaaccagugcucaaaggagucaaacuccauuacacuuga

(19) United States
(12) Patent Application Publication
(10) Pub. No.: US 2023/0355741 A1
(43) Pub. Date: Nov. 9, 2023

(54) Feline Severe Acute Respiratory Syndrome Coronavirus 2 Vaccine

(71) Applicant: Intervet Inc., Madison, NJ (US)

(72) Inventors: Mark A. Mogler, Ames, IA (US); Ian Tarpey, St. Ives (GB)

(73) Assignee: Intervet Inc., Madison, NJ (US)

(21) Appl. No.: 17/923,018

(22) PCT Filed: May 10, 2021
(86) PCT No.: PCT/EP2021/062246

§ 371 Date: Nov. 3, 2022

Related U.S. Application Data
(60) Provisional application No. 63/022,775, filed on May 11, 2020.

(51) Int. Cl. A61K 39/215; A61K 39/205; A61K 39/21; A61K 39/12; A61K 39/23; A61K 39/175; A61K 39/145; A61K 39/118; A61P 31/14; C12N 7/00

(52) U.S. Cl. CPC A61K 39/215; A61K 39/205; A61K 39/21; A61K 39/12; A61K 39/23; A61K 39/175; A61K 39/145; A61K 39/118; A61P 31/14; C12N 7/00; A61K 2039/5256

(57) ABSTRACT

The present invention provides new vaccines for felines and ferrets to aid in reducing shedding of severe acute respiratory syndrome coronavirus 2 by infected felines or ferrets. Methods of making and using the vaccines alone or in combinations with other protective agents are also provided.

### CROSS-REFERENCE TO RELATED APPLICATIONS

This application claims priority under 35 U.S.C. § 119(e) of provisional application U.S. Ser. No. 63/022,775, filed May 11, 2020. The contents of which are hereby incorporated by reference in their entireties.

### FIELD OF THE INVENTION

The present invention relates to new vaccines for felines and ferrets to aid in reducing shedding of severe acute respiratory syndrome coronavirus 2 by felines or ferrets. Methods of making and using the vaccines alone or in combinations with other protective agents are also provided.

### BACKGROUND OF THE INVENTION

The etiological agent of the worldwide pandemic of 2019-2020, universally referred to as Coronavirus Disease 2019 (COVID-19), is an extremely contagious respiratory (and possibly also enteric) coronavirus named Severe Acute Respiratory Syndrome Coronavirus 2 (SARS-20 CoV-2). SARS-CoV-2 is member of the Coronaviridae family, of the order Nidovirales, and the genus Betacoronavirus. Although coronavirus infections in humans had been reported in the past century, they generally resulted in common cold-like symptoms, whereas SARS-CoV-2 follows the 2003 SARS epidemic (SARS-CoV) and the 2012 Middle East Respiratory Syndrome coronavirus (MERS-CoV) as the third major Betacoronavirus outbreak of the present millennium.

Coronaviruses are enveloped, single stranded, non-segmented, positive sense RNA viruses that encode sixteen non-structural proteins, several accessory proteins, and four major structural proteins: the spike surface protein (S protein), which is a large glycoprotein protruding from the surface of the virus; (ii) an integral membrane (or matrix) protein (M); (iii) a small membrane envelope protein (E); and (iv) a nucleocapsid protein (N). The spike protein of a coronavirus determines the tropism of the coronavirus by binding to a specific extracellular domain of a host target protein that spans the membrane of the host cells of the infected animal. The target protein is denoted as the receptor. The receptor for both SARS-CoV and SARS-CoV-2 is the angiotensin-converting enzyme 2 (ACE2), a type I integral membrane protein that is a zinc metalloenzyme that functions as a monocarboxypeptidase and plays an important role in vascular health. The primary function of ACE2 is to counterbalance the effect of the angiotensin-converting enzyme (ACE). ACE cleaves the angiotensin I hormone into the vasoconstricting peptide angiotensin II, whereas ACE2 cleaves the C-terminal amino acid of angiotensin II, ultimately resulting in the formation of a counter-acting vasodilating peptide. The binding of the spike protein of SARS-CoV-2 to ACE2 results in endocytosis and translocation of the virus into endosomes located within cells.

SARS-CoV-2 is thought to have zoonotic origins, with SARS-CoV-2 evolving from a bat coronavirus (bat CoV), either directly or through an intermediary animal [Wu et al., Cell Host & Microbe 27:1-4 (Mar. 11, 2020)]. Indeed, both SARS-CoV and SARS-CoV-2 are believed to have come from different SARS-like bat CoVs, both potentially with intermediary hosts. It has been suggested that SARS-CoV made its way to humans from bats via captive Himalayan palm civets (Paguma larvata) [Wu et al., Cell Host & Microbe 27:1-4 (Mar. 11, 2020); Guan et al., Science 302: 276-278 (2003)]. Notably, Himalayan palm civets also have been shown to be extremely susceptible to SARS-CoV [Kan et al., J. of Virol. 79(18):11892-11900 (2005); Guan et al., Science 302: 276-278 (2003)]. Consistently, comparing the nucleotide sequences of their entire genomes indicates that SARS-CoV-2 is genetically more closely related to SARS-like bat CoVs than to SARS-CoV [Wu et al., Cell Host & Microbe 27:1-4 (Mar. 11, 2020)].

As with SARS-CoV, there have been a number of reports in the general media of lions and tigers in zoos, as well of domestic cats, testing positive for SARS-CoV-2. Some of these felines, including domestic cats, have demonstrated clinical signs of infection and significant post-mortem lung lesions. Recent reports also have shown that SARS-CoV-2 can infect ferrets, hamsters, and mink.

Although, to date, there has been no reports of humans contracting COVID-19 from domestic cats, it remains a great fear that such a transmission could occur. One basis for this fear comes from studies that report that infected domestic cats may shed sufficient SARS-CoV-2 by aerosol to infect other cats that have been kept physically distant. Furthermore, based on their rate of seroconversion, studies suggest that cat to cat transmission of SARS-CoV-2 may occur in a natural setting.

Somewhat encouraging, in preliminary studies SARS-CoV-2 has shown relatively modest genetic diversity, suggesting that the right feline vaccine against SARS-CoV-2 may be successful. Ideally, such a vaccine would prevent transmission of the virus to cats, prevent cats from becoming a reservoir for the virus, and/or reduce the shedding of SARS-CoV-2 by infected cats. Currently, there are over 100 potential SARS-CoV-2 vaccines being developed for humans, with researchers employing many different vaccine strategies. Currently there are several SARS-CoV-2 for humans, which are hoped to be significant in countering the spread or effect of COVID-19.

The use of alphavirus-derived replicon RNA particles (RP) is one of the large number of vector strategies that have been employed in vaccines through the years to protect against specific animal pathogens [Vander Veen, et al. Anim Health Res Rev. 13(1):1-9. (2012) doi: 10.1017/S1466252312000011; Kamrud et al., J Gen Virol. 91(Pt 7):1723-1727 (2010)]. Alphavirus-derived RPs have been developed for several different alphaviruses, including Venezuelan equine encephalitis virus (VEE) [Pushko et al., Virology 239:389-401 (1997)], Sindbis (SIN) [Bredenbeek et al., Journal of Virology 67:6439-6446 (1993)], and Semliki Forest virus (SFV) [Liljestrom and Garoff, Biotechnology (NY) 9:1356- 1361 (1991)]. RP vaccines deliver propagation-defective alphavirus RNA replicons into host cells and result in the expression of the desired immunogenic transgene(s) in vivo [Pushko et al., Virology 239(2):389-401 (1997)]. The construction of a hybrid VEE/SIN replication particle encoding the SARS-CoV spike protein that expresses detectable spike protein, in vitro, has been reported [U.S. Pat. No. 9,730,997]. RPs also have an attractive safety and efficacy profile when compared to some traditional vaccine formulations [Vander Veen, et al. Anim Health Res Rev. 13(1):1-9 (2012)]. Furthermore, the VEE RP platform has been used to encode pathogenic antigens from canines and felines [see e.g., WO2019/086645, WO2019/086646, and WO2019/115090] and is the basis for several USDA-licensed vaccines for swine and poultry.

The citation of any reference herein should not be construed as an admission that such reference is available as “prior art” to the instant application.

### SUMMARY OF THE INVENTION

Accordingly, the present invention provides Venezuelan Equine Encephalitis (VEE) alphavirus RNA replicon particles that encode one or more SARS-CoV-2 protein antigens. Such vectors can be used in immunogenic compositions comprising these RNA vectors. The immunogenic compositions of the present invention may be used in vaccines.

In one aspect, the present invention provides an immunogenic composition comprising a Venezuelan Equine Encephalitis (VEE) alphavirus RNA replicon particle that encodes a SARS-CoV-2 protein antigen. In particular embodiments of this type, the SARS-CoV-2 protein antigen is the SARS-CoV-2 spike protein or an immunogenic fragment thereof In specific embodiments of this type, the SARS-CoV-2 spike protein comprises an amino acid sequence comprising at least 95% identity with the amino acid sequence of SEQ ID NO: 2. In more specific embodiments of this type, the SARS-CoV-2 spike protein comprises an amino acid sequence comprising at least 98% identity with the amino acid sequence of SEQ ID NO: 2. In even more specific embodiments of this type, the SARS-CoV-2 spike protein comprises an amino acid sequence comprising at least 99% identity with the amino acid sequence of SEQ ID NO: 2. In still more specific embodiments of this type, the SARS-CoV-2 spike protein comprises an amino acid sequence comprising 99.5% or greater identity with the amino acid sequence of SEQ ID NO: 2. In yet more specific embodiments of this type, the SARS-CoV-2 spike protein comprises an amino acid sequence comprising the amino acid sequence of SEQ ID NO: 2.

In related embodiments of this type, the SARS-CoV-2 spike protein is encoded by a nucleotide sequence comprising at least 95% identity with the nucleotide sequence of SEQ ID NO: 4. In more specific embodiments of this type, the SARS-CoV-2 spike protein is encoded by a nucleotide sequence comprising at least 98% identity with the nucleotide sequence of SEQ ID NO: 4. In even more specific embodiments of this type, the SARS-CoV-2 spike protein is encoded by a nucleotide sequence comprising at least 99% identity with the nucleotide sequence of SEQ ID NO: 4. In still more specific embodiments of this type, the SARS-CoV-2 spike protein is encoded by a nucleotide sequence comprising at least 99.5% identity with the nucleotide sequence of SEQ ID NO: 4. In yet more specific embodiments of this type, the SARS-CoV-2 spike protein is encoded by a nucleotide sequence by the nucleotide sequence of SEQ ID NO: 4.

In another aspect, the present invention provides an immunogenic composition comprising a VEE alphavirus RNA replicon particle that encodes two or more antigens with the first antigen being a SARS-CoV-2 protein antigen. In particular embodiments of this type, the first SARS-CoV-2 protein antigen is the SARS-CoV-2 spike protein or an immunogenic fragment thereof. In specific embodiments of this type, the first SARS-CoV-2 spike protein comprises an amino acid sequence comprising at least 95% identity with the amino acid sequence of SEQ ID NO: 2. In more specific embodiments of this type, the first SARS-CoV-2 spike protein comprises an amino acid sequence comprising at least 98% identity with the amino acid sequence of SEQ ID NO: 2. In even more specific embodiments of this type, the first SARS-CoV-2 spike protein comprises an amino acid sequence comprising at least 99% identity with the amino acid sequence of SEQ ID NO: 2. In still more specific embodiments of this type, the first SARS-CoV-2 spike protein comprises an amino acid sequence comprising 99.5% or greater identity with the amino acid sequence of SEQ ID NO: 2. In yet more specific embodiments of this type, the first SARS-CoV-2 spike protein comprises an amino acid sequence comprising the amino acid sequence of SEQ ID NO: 2.

In related embodiments of this type, the SARS-CoV-2 spike protein is encoded by a nucleotide sequence comprising at least 95% identity with the nucleotide sequence of SEQ ID NO: 4. In more specific embodiments of this type, the SARS-CoV-2 spike protein is encoded by a nucleotide sequence comprising at least 98% identity with the nucleotide sequence of SEQ ID NO: 4. In even more specific embodiments of this type, the SARS-CoV-2 spike protein is encoded by a nucleotide sequence comprising at least 99% identity with the nucleotide sequence of SEQ ID NO: 4. In still more specific embodiments of this type, the SARS-CoV-2 spike protein is encoded by a nucleotide sequence comprising at least 99.5% identity with the nucleotide sequence of SEQ ID NO: 4. In yet more specific embodiments of this type, the SARS-CoV-2 spike protein is encoded by a nucleotide sequence by the nucleotide sequence of SEQ ID NO: 4.

In certain embodiments, the VEE alphavirus RNA replicon particles further encode one or more other antigens. In particular embodiments of this type, the VEE alphavirus RNA replicon particles further encode a second SARS-CoV-2 protein antigen. In more particular embodiments, the second SARS-CoV-2 protein antigen is a second SARS-CoV-2 spike protein that originates from a different strain of SARS-CoV-2 than the first SARS-CoV-2 spike protein originates from. In other embodiments, the VEE alphavirus RNA replicon particles encode the first SARS-CoV-2 spike protein, optionally together with the second SARS-CoV-2 spike protein, and an antigen from a non-SARS-CoV-2. In still other embodiments, the non-SARS-CoV-2 antigen is a feline calicivirus (FCV) capsid protein. In yet other embodiments the non-SARS-CoV-2 antigen is a rabies virus glycoprotein (G). In still other embodiments, the non-SARS-CoV-2 antigen is feline leukemia virus (FeLV) envelope protein.

In related embodiments, the present invention provides immunogenic composition comprising one or more additional VEE alphavirus RNA replicon particles, which encode a second SARS-CoV-2 protein antigen. In particular embodiments, a first VEE alphavirus RNA replicon particle encodes a first SARS-CoV-2 spike protein and a second VEE alphavirus RNA replicon particle encodes a second SARS-CoV-2 spike protein that originates from a different strain of SARS-CoV-2 than the first SARS-CoV-2 spike protein originates from. In specific embodiments of this type, the first SARS-CoV-2 spike protein comprises an amino acid sequence comprising at least 95% identity with the amino acid sequence of SEQ ID NO: 2. In more specific embodiments of this type, the first SARS-CoV-2 spike protein comprises an amino acid sequence comprising at least 98% identity with the amino acid sequence of SEQ ID NO: 2. In even more specific embodiments of this type, the first SARS-CoV-2 spike protein comprises an amino acid sequence comprising at least 99% identity with the amino acid sequence of SEQ ID NO: 2. In still more specific embodiments of this type, the first SARS-CoV-2 spike protein comprises an amino acid sequence comprising 99.5% or greater identity with the amino acid sequence of SEQ ID NO: 2. In yet more specific embodiments of this type, the first SARS-CoV-2 spike protein comprises an amino acid sequence comprising the amino acid sequence of SEQ ID NO: 2.

In related embodiments of this type, the SARS-CoV-2 spike protein is encoded by a nucleotide sequence comprising at least 95% identity with the nucleotide sequence of SEQ ID NO: 4. In more specific embodiments of this type, the SARS-CoV-2 spike protein is encoded by a nucleotide sequence comprising at least 98% identity with the nucleotide sequence of SEQ ID NO: 4. In even more specific embodiments of this type, the SARS-CoV-2 spike protein is encoded by a nucleotide sequence comprising at least 99% identity with the nucleotide sequence of SEQ ID NO: 4. In still more specific embodiments of this type, the SARS-CoV-2 spike protein is encoded by a nucleotide sequence comprising at least 99.5% identity with the nucleotide sequence of SEQ ID NO: 4. In yet more specific embodiments of this type, the SARS-CoV-2 spike protein is encoded by a nucleotide sequence by the nucleotide sequence of SEQ ID NO: 4.

The present invention further provides vaccines that comprise an immunogenic composition of the present invention and a pharmaceutically acceptable carrier to aid in reducing shedding of SARS-CoV-2 in a feline. The present invention further provides vaccines that comprise an immunogenic composition of the present invention and a pharmaceutically acceptable carrier to aid in reducing shedding of SARS-CoV-2 in a ferret. In other embodiments, the feline vaccines aid in reducing the severity of one or more clinical signs in the infected feline. In still other embodiments the ferret vaccines aid in reducing the severity of one or more clinical signs in the infected ferrets.

The vaccines of the present invention can further comprise at least one non-SARS-CoV-2 antigen for eliciting protective immunity to a non-SARS-CoV-2 pathogen. In certain embodiments of this type, vaccines further comprise a VEE alphavirus RNA replicon particle comprising a nucleotide sequence encoding at least one antigen or immunogenic fragment thereof that originates from the non-SARS-CoV-2 pathogen. In alternative vaccine embodiments, the non-SARS-CoV-2 antigen is an inactivated non-SARS-CoV-2 pathogen. In still other embodiments, the non-SARS-CoV-2 antigen is an attenuated non-SARS-CoV-2 pathogen.

In particular vaccine embodiments, the non-SARS-CoV-2 pathogen is a feline calicivirus (FCV). In other vaccine embodiments, the non-SARS-CoV-2 pathogen is a feline leukemia virus (FeLV). In yet other vaccine embodiments, the non-SARS-CoV-2 pathogen is a feline panleukopenia virus (FPLV). In still other vaccine embodiments, the non-SARS-CoV-2 pathogen is a feline rhinotracheitis virus (FVR). In yet other vaccine embodiments, the non-SARS-CoV-2 pathogen is a Chlamydophila felis. In still other vaccine embodiments, the non-SARS-CoV-2 pathogen is a canine influenza virus (CIV). In yet other vaccine embodiments, the non-SARS-CoV-2 pathogen is a canine parvovirus (CPV). In still other vaccine embodiments, the non-SARS-CoV-2 pathogen is a canine distemper (CDV). In yet other vaccine embodiments, the non-SARS-CoV-2 pathogen is a rabies virus. In certain vaccine embodiments, the vaccines comprise non-SARS-CoV-2 antigens from multiple non-SARS-CoV-2 pathogens. In related embodiments of the feline or ferret vaccines, the non-SARS-CoV-2 pathogen is selected from one or more of FCV, FeLV, FPLV, FVR, CIV, CPV, CDV, rabies virus. In more particular embodiments, the vaccine is a feline vaccine and the multiple non-SARS-CoV-2 pathogens are selected from one or more of FCV, FeLV, FPLV, FVR, CIV, rabies virus, and Chlamydophila felis or any combination thereof In related embodiments, the vaccine is a ferret vaccine and the non-SARS-CoV-2 pathogen is selected from the group consisting of CPV, CDV, and the combination thereof.

Immunogenic compositions and/or vaccines (including multivalent vaccines) comprising a recombinant vector of the present invention, e.g., an alphavirus RNA replicon particle encoding a SARS-CoV-2 spike protein can be administered in the presence, or alternatively, in the absence of an adjuvant. Accordingly, in certain embodiments of the invention, a vaccine may not comprise an adjuvant and accordingly, is a non-adjuvanted vaccine.

Alternatively, in specific embodiments, the vaccine of the present invention does comprise an adjuvant. In particular embodiments of this type, the adjuvant comprises polymers of acrylic acid cross-linked with polyalkenyl ethers or divinyl glycol [e.g., CARBOPOLI®]. In other embodiments the adjuvant comprises Alhydrogel and QuilA. In related embodiments the adjuvant comprises Alhydrogel, saponin (e.g., QS21), and Carbigen. In still other embodiments the adjuvant comprises aluminium hydroxide. In yet other embodiments the adjuvant comprises Adjuphos. In still other embodiments the adjuvant comprises Emulsigen, EMA31, and Neocryl XK62.

The present invention further provides methods of immunizing a mammal against SARS-CoV-2 comprising administering to the mammal an immunologically effective amount of a vaccine of the present invention. In certain embodiments, the mammal is a feline. In particular embodiments, the feline is a domestic cat. In still other embodiments the feline is a lion. In still other embodiments the feline is a tiger. In related embodiments, the mammal is a ferret.

These and other aspects of the present invention will be better appreciated by reference to the following Brief Description of the Drawings and the Detailed Description.

### BRIEF DESCRIPTION OF THE DRAWINGS

FIGS. 1A-1C show the immunogenicity of SARS-CoV-2 Spike antigen in guinea pigs. FIG. 1A depicts the SARS-CoV-2 Surrogate VN test assay results of 1,000-fold diluted serum taken at different days post vaccination (d.p.v.) from animals vaccinated with VEEV Replicon Particles (black circle) or Plasmid DNA (open circles) vaccines. FIG. 1B depicts the spike ectodomain ELISA using serum taken at different days post vaccination (d.p.v.) from animals vaccinated with VEEV Replicon Particles (black circle) or Plasmid DNA (open circles) vaccines. FIG. 1C depicts the lymphocyte stimulation test (LST) from blood collected on day 70/71. Purified SARS-CoV-2 S1 antigen was used to stimulate isolated lymphocytes and proliferation was measured 96 hours after stimulation.

### DETAILED DESCRIPTION OF THE INVENTION

The present invention provides immunogenic compositions and vaccines that could aid in the prevention of, or even prevent, disease in cats and ferrets caused by SARS-CoV-2. These vaccines may not only be beneficial to the vaccinated cats and ferrets, but also may prevent them from becoming a reservoir for the virus, where further unknown and potentially deleterious mutations could arise. Moreover, such vaccines could lead to the reduction or even elimination of the viral shed of SARS-CoV-2 in cats and ferrets. Such viral shed could result in the transmission of SARS-CoV-2 to other animals, including humans.

Accordingly, the present invention provides immunogenic compositions and/or vaccines (including multivalent vaccines) that encode a protein antigen from SARS-CoV-2 (e.g., SARS-CoV-2 spike protein). In one aspect of the present invention, the vaccines comprise alphavirus RNA replicon particles (RPs) that comprise the capsid protein and glycoproteins of Venezuelan Equine Encephalitis Virus (VEE) and encode a protein antigen or immunogenic fragment thereof from SARS-CoV-2. In even more specific embodiments, the vaccines comprise alphavirus RNA replicon particles (RPs) that comprise the capsid protein and glycoproteins of the avirulent TC-83 strain of VEE and encode a protein antigen or immunogenic fragment thereof from SARS-CoV-2. Immunogenic compositions and/or vaccines (including multivalent vaccines) comprising the alphavirus RNA replicon particles encoding a protein antigen or immunogenic fragment thereof from SARS-CoV-2 can be administered in the presence or alternatively in the absence of an adjuvant. In particular embodiments, the antigen is the SARS-CoV-2 spike protein. In certain embodiments, the immunogenic compositions and/or vaccines are for felines. In other embodiments, the immunogenic compositions and/or vaccines are for ferrets. Methods of making and using the vaccines and/or immunogenic compositions alone or in combinations with other protective agents are also provided.

In order to more fully appreciate the invention, the following definitions are provided.

The use of singular terms for convenience in description is in no way intended to be so limiting. Thus, for example, reference to a composition comprising “a polypeptide” includes reference to one or more of such polypeptides. In addition, reference to an “alphavirus RNA replicon particle” includes reference to a plurality of such alphavirus RNA replicon particles, unless otherwise indicated.

As used herein the term “approximately” is used interchangeably with the term “about” and signifies that a value is within fifty percent of the indicated value i.e., a composition containing “approximately” 1×10⁸ alphavirus RNA replicon particles per milliliter contains from 5×10⁷ to 1.5×10⁸ alphavirus RNA replicon particles per milliliter.

As used herein, the term “canine” includes all domestic dogs, Canis lupus familiaris or Canis familiaris, unless otherwise indicated.

As used herein, the term “feline” refers to any member of the Felidae family. Members of this family include wild, zoo, and domestic members, such as any member of the subfamilies Felinae, e.g., cats, lions, tigers, pumas, jaguars, leopards, snow leopards, panthers, North American mountain lions, cheetahs, lynx, bobcats, caracals or any cross breeds thereof. Cats also include domestic cats (Felis catus) including pure-bred and/or mongrel companion cats, show cats, laboratory cats, cloned cats, and wild or feral cats.

As used herein, a “ferret” is a mammal that is one of the mammals that belong to the mustelid family.

As used herein, the term “replicon” refers to a modified RNA viral genome that lacks one or more elements (e.g., coding sequences for structural proteins) that if they were present, would enable the successful propagation of the parental virus in cell cultures or animal hosts. In suitable cellular contexts, the replicon will amplify itself and may produce one or more sub-genomic RNA species.

As used herein, the term “alphavirus RNA replicon particle”, abbreviated “RP”, is an alphavirus-derived RNA replicon packaged in structural proteins, e.g., the capsid and glycoproteins, which also are derived from an alphavirus, e.g., as described by Pushko et al., [Virology 239(2):389-401 (1997)]. An RP cannot propagate in cell cultures or animal hosts (without a helper plasmid or analogous component), because the replicon does not encode the alphavirus structural components (e.g., capsid and glycoproteins).

The terms “originate from”, “originates from” and “originating from” are used interchangeably with respect to a given protein antigen and the pathogen or strain of that pathogen that naturally encodes it, and as used herein signify that the unmodified and/or truncated amino acid sequence of that given protein antigen or immunogenic fragment thereof is encoded by that pathogen or strain of that pathogen. The coding sequence, within a nucleic acid construct of the present invention for a protein antigen originating from a pathogen may have been genetically manipulated so as to result in a modification and/or truncation of the amino acid sequence of the expressed protein antigen relative to the corresponding sequence of that protein antigen in the pathogen or strain of pathogen (including naturally attenuated strains) it originates from.

The spike protein of a Coronavirus is a large glycoprotein protruding from the surface of the virus that determines the tropism of the virus by binding to a specific extracellular domain of a host receptor. Human angiotensin-converting enzyme 2 (ACE2) serves as the host receptor for both the SARS-CoV-2 and the SARS-CoV spike proteins. The most variable part of the coronavirus genome is the receptor binding domain (RBD) of coronavirus spike proteins. Notably however, five of the six critical amino acid residues of the RBD differ between the SARS-CoV-2 spike protein and the SARS-CoV spike protein. The SARS-CoV-2 spike protein further differs from a SARS-CoV spike protein by the SARS-CoV-2 spike protein comprising a polybasic cleavage site (RRAR) at the junction of the spike protein's two subunits, Si and S2, whereas the SARS-CoV spike protein does not [see, Andersen et al., Nature Medicine 26:450-455 (2020)]. This polybasic cleavage site allows effective cleavage by proteases, which plays a role in the infectivity of SARS-CoV-2. Although the polybasic cleavage site is not unique to the SARS-CoV-2 spike protein, as the spike proteins of some of other human beta coronaviruses comprise such structures, like SARS-CoV, the spike protein of the most closely related bat coronaviruses also have not been found to comprise this polybasic cleavage site.

The term “non-SARS-CoV-2”, is used to modify terms such as pathogen, and/or antigen or immunogenic fragment thereof to signify that the respective pathogen, and/or antigen is neither a SARS-CoV-2 nor a SARS-CoV-2 protein antigen or immunogenic fragment thereof. A non-SARS-CoV-2 antigen does not originate from a SARS-CoV-2.

As used herein, the terms “modified live” and “attenuated” are used interchangeably with respect to a given live virus and/or a live micro-organism.

As used herein, the terms “protecting”, and/or “providing protection to”, and/or “eliciting protective immunity to”, and/or “aids in the prevention of a disease”, and/or “aids in the protection”, and/or “reduces viral load”, and/or “reduces viremia” do not require complete protection from any indication of infection. For example, “aids in the protection” can mean that the protection is sufficient such that, after challenge, symptoms of the underlying infection are at least reduced, and/or aid in the reduction of viral shedding, and/or that one or more of the underlying cellular, physiological, or biochemical causes or mechanisms causing the symptoms are reduced and/or eliminated. It is understood that “reduced,” as used in this context, means relative to the state of the infection, including the molecular state of the infection, not just the physiological state of the infection.

As used herein, a “vaccine” is a composition that is suitable for application to an animal, e.g., a feline (including, in certain embodiments, humans, while in other embodiments being specifically not for humans) comprising one or more antigens typically combined with a pharmaceutically acceptable carrier such as a liquid containing water, which upon administration to the animal induces an immune response strong enough to minimally aid in the protection from a disease arising from an infection with a wild-type virus and/or wild-type micro-organism, i.e., strong enough for aiding in the prevention of the disease, and/or preventing, ameliorating or curing the disease.

As used herein, a multivalent vaccine is a vaccine that comprises two or more different antigens. In a particular embodiment of this type, the multivalent vaccine stimulates the immune system of the recipient against two or more different pathogens.

The terms “adjuvant” and “immune stimulant” are used interchangeably herein and are defined as one or more substances that cause stimulation of the immune system. In this context, an adjuvant is used to enhance an immune response to one or more vaccine antigens/isolates. Accordingly, “adjuvants” are agents that nonspecifically increase an immune response to a particular antigen, thus reducing the quantity of antigen necessary in any given vaccine, and/or the frequency of injection necessary in order to generate an adequate immune response to the antigen of interest. In this context, an adjuvant is used to enhance an immune response to one or more vaccine antigens/isolates.

As used herein, a “nonadjuvanted vaccine” is a vaccine or a multivalent vaccine that does not contain an adjuvant.

As used herein, the term “pharmaceutically acceptable” is used adjectivally to mean that the modified noun is appropriate for use in a pharmaceutical product. When it is used, for example, to describe an excipient in a pharmaceutical vaccine, it characterizes the excipient as being compatible with the other ingredients of the composition and not disadvantageously deleterious to the intended recipient animal, e.g., a feline.

The term “carrier” refers to a diluent, adjuvant, excipient, or vehicle with which the alphavirus RNA replicon particles are administered. Pharmaceutical acceptable carriers can be sterile liquids, such as water and/or oils, including those of petroleum, animal, vegetable or synthetic origin, such as peanut oil, soybean oil, mineral oil, sesame oil and the like. Water or aqueous solution saline solutions and aqueous sugar, e.g., dextrose and/or glycerol solutions can be employed as carriers, particularly for injectable solutions. In the case of nonadjuvanted vaccines, the carrier cannot be an adjuvant.

“Parenteral administration” includes subcutaneous injections, submucosal injections, intravenous injections, intramuscular injections, intradermal injections, oral, intranasal, and infusion.

As used herein the term “immunogenic fragment” in regard to a particular protein (e.g., a protein antigen) is a fragment of that protein that is immunogenic, i.e., capable of specifically interacting with an antigen recognition molecule of the immune system, such as an immunoglobulin (antibody) or T cell antigen receptor. Preferably, an immunogenic fragment of the present invention is immunodominant for antibody and/or T cell receptor recognition. In particular embodiments, an immunogenic fragment with respect to a given protein antigen is a fragment of that protein that retains at least 25% of the antigenicity of the full-length protein SARS-CoV-2 spike protein. In preferred embodiments an immunogenic fragment retains at least 50% of the antigenicity of the full-length protein SARS-CoV-2 spike protein. In more preferred embodiments, an immunogenic fragment retains at least 75% of the antigenicity of the full-length protein SARS-CoV-2 spike protein. Immunogenic fragments can be 100 amino acids or more that comprise at least one conserved region of the full-length protein SARS-CoV-2 spike protein or at the other extreme, be large fragments that are missing as little as a single amino acid from the full-length protein. In particular embodiments, the immunogenic fragment comprises 125 to 1000 amino acid residues of the full-length protein SARS-CoV-2 spike protein. In other embodiments, the immunogenic fragment comprises 250 to 750 amino acid residues of the full-length protein SARS-CoV-2 spike protein.

As used herein one amino acid sequence is 100% “identical” or has 100% “identity” to a second amino acid sequence when the amino acid residues of both sequences are identical. Accordingly, an amino acid sequence is 50% “identical” to a second amino acid sequence when 50% of the amino acid residues of the two amino acid sequences are identical. The sequence comparison is performed over a contiguous block of amino acid residues comprised by a given protein, e.g., a protein, or a portion of the polypeptide being compared. In a particular embodiment, selected deletions or insertions that could otherwise alter the correspondence between the two amino acid sequences are taken into account.

As used herein, nucleotide and amino acid sequence percent identity can be determined using C, MacVector (MacVector, Inc. Cary, NC 27519), Vector NTI (Informax, Inc. MD), Oxford Molecular Group PLC (1996) and the Clustal W algorithm with the alignment default parameters, and default parameters for identity. These commercially available programs can also be used to determine sequence similarity using the same or analogous default parameters. Alternatively, an Advanced Blast search under the default filter conditions can be used, e.g., using the GCG (Genetics Computer Group, Program Manual for the GCG Package, Version 7, Madison, Wisconsin) pileup program using the default parameters.

For the purposes of this invention, an “inactivated” virus or microorganism is a virus or micro-organism which is capable of eliciting an immune response in an animal, but is not capable of infecting the animal. For example, an inactivated SARS-CoV-2 may be inactivated by an agent selected from the group consisting of binary ethyleneimine, formalin, beta-propiolactone, thimerosal, or heat.

The alphavirus RNA replicon particles of the present invention may be lyophilized and rehydrated with a sterile water diluent. On the other hand, when the alphavirus RNA replicon particles are stored separately, but intended to be mixed with other vaccine components prior to administration, the alphavirus RNA replicon particles can be stored in the stabilizing solution of those components, e.g., a high sucrose solution.

In one aspect of the present invention, the vaccines are non-adjuvanted, i.e., do not comprise an adjuvant. On the other hand, in certain embodiments the vaccines do contain an adjuvant. Examples of adjuvants that may be used in the vaccines of the present invention include CARBOPOL® [e.g., polymers of acrylic acid cross-linked with polyalkenyl ethers or divinyl glycol], Alhydrogel+QuilA, aluminium hydroxide, Alhydrogel, Emulsigen+EMA31+Neocryl XK62, Carbomer, Carbomer 974P, Adjuphos, and Alhydrogel+QS21 (saponin) Carbigen.

A vaccine of the present invention can be readily administered by any standard route including intravenous, intramuscular, subcutaneous, oral, intranasal, intradermal, and/or intraperitoneal vaccination. The artisan will appreciate that the vaccine composition is preferably formulated appropriately for each type of recipient animal and route of administration. Thus, the present invention also provides methods of immunizing a mammal against SARS-CoV-2 and/or other mammalian pathogens. One such method comprises injecting a mammal with an immunologically effective amount of a vaccine of the present invention, so that the mammal produces appropriate antibodies to the SARS-CoV-2 spike protein.

### Multivalent Vaccines

The present invention also provides multivalent vaccines. Any antigen or combination of such antigens useful in a mammalian vaccine can be added to a propagation defective alphavirus RNA replicon particle (RP) that encodes an antigen of the SARS-CoV-2 [e.g., the SARS-CoV-2 spike protein] in the vaccine. Accordingly, such multivalent vaccines are included in the present invention. Examples of pathogens that one or more of such protein antigens can originate for the feline or ferret immunogenic compositions and vaccines include feline calicivirus, feline rhinotracheitis Virus (FVR), feline leukemia virus (FeLV), feline panleukopenia Virus (FPL), feline immunodeficiency (Hy), rabies virus, canine influenza virus, canine parvovirus, canine distemper virus, and feline chlamydia.

In addition, an alphavirus RNA replicon particle(RP) that encodes one or more antigens of the SARS-CoV-2 [e.g., the SARS-CoV-2 spike protein or immunogenic fragment thereof] can be added together with one or more other live, attenuated virus isolates, e.g., a live attenuated FCV virus and/or a live, attenuated feline leukemia virus, and/or a live, attenuated feline infectious peritonitis virus and/or a live, attenuated feline immunodeficiency virus, and/or a live, attenuated rabies virus, and/or a live, attenuated feline influenza virus and/or a live, attenuated canine influenza virus. In addition, a live, attenuated Chlamydophila felis, and/or a live, attenuated Bordetella bronchiseptica and/or a live, attenuated Bartonella spp. (e.g., B. henselae) can also be included in such multivalent vaccines.

Furthermore, an alphavirus RNA replicon particle (RP) that encodes one or more protein antigens of SARS-CoV-2 (e.g., the SARS-CoV-2 spike protein or an antigenic fragment thereof) can be added together with one or more other inactivated virus isolates such as an inactivated FCV strain, and/or an inactivated feline herpesvirus and/or an inactivated feline parvovirus and/or an inactivated feline leukemia virus, and/or an inactivated feline infectious peritonitis virus and/or an inactivated feline immunodeficiency virus, and/or an inactivated rabies virus, and/or an inactivated feline influenza virus, and/or an inactivated canine influenza virus. In addition, bacterins (or subfractions of the bacterins, e.g., the pilus subfraction) of Chlamydophila felis, and/or Bordetella bronchiseptica and/or Bartonella spp. (e.g., B. henselae) can also be included in such multivalent vaccines.

### SEQUENCE TABLE

| SEQ ID | | |
|---|---|---|
| NO: | Description | Type |
| 1 | Wild-type SARS-CoV-2 Spike Protein | DNA |
| 2 | Wild-type SARS-CoV-2 Spike Protein | Amino Acid |
| 3 | Wild-type SARS-CoV-2 Spike Protein | DNA |
| | Codon-optimized | |
| 4 | Wild-type SARS-CoV-2 Spike Protein | RNA |
| | Codon-optimized | |

### SEQUENCES

Severe acute respiratory syndrome coronavirus 2 isolate 2019-nCoV/U.S.A.-WI1/2020, complete genome; GenBank:

MT039887.1 MT039887_Wild-type_Spike_nucleotide:
SEQ ID NO: 1
atgtttgtttttcttgttttattgccactagtctctagtcagtgtgttaatcttacaaccagaactcaat
taccccctgcatacactaattctttcacacgtggtgtttattaccctgacaaagttttcagatcctcagt
tttacattcaactcaggacttgttcttacctttcttttccaatgttacttggttccatgctatacatgtc
tctgggaccaatggtactaagaggtttgataaccctgtcctaccatttaatgatggtgtttattttgctt
ccactgagaagtctaacataataagaggctggatttttggtactactttagattcgaagacccagtccct
acttattgttaataacgctactaatgttgttattaaagtctgtgaatttcaattttgtaatgatccattt
ttgggtgtttattaccacaaaaacaacaaaagttggatggaaagtgagttcagagtttattctagtgcga
ataattgcacttttgaatatgtctctcagccttttcttatggaccttgaaggaaaacagggtaatttcaa
aaatcttagggaatttgtgtttaagaatattgatggttattttaaaatatattctaagcacacgcctatt
aatttagtgcgtgatctccctcagggtttttcggctttagaaccattggtagatttgccaataggtatta
acatcactaggtttcaaactttacttgctttacatagaagttatttgactcctggtgattcttcttcagg
ttggacagctggtgctgcagcttattatgtgggttatcttcaacctaggacttttctattaaaatataat
gaaaatggaaccattacagatgctgtagactgtgcacttgaccctctctcagaaacaaagtgtacgttga
aatccttcactgtagaaaaaggaatctatcaaacttctaactttagagtccaaccaacagaatctattgt
tagatttcctaatattacaaacttgtgcccttttggtgaagtttttaacgccaccagatttgcatctgtt
tatgcttggaacaggaagagaatcagcaactgtgttgctgattattctgtcctatataattccgcatcat
tttccacttttaagtgttatggagtgtctcctactaaattaaatgatctctgctttactaatgtctatgc
agattcatttgtaattagaggtgatgaagtcagacaaatcgctccagggcaaactggaaagattgctgat
tataattataaattaccagatgattttacaggctgcgttatagcttggaattctaacaatcttgattcta
aggttggtggtaattataattacctgtatagattgtttaggaagtctaatctcaaaccttttgagagaga
tatttcaactgaaatctatcaggccggtagcacaccttgtaatggtgttgaaggttttaattgttacttt
cctttacaatcatatggtttccaacccactaatggtgttggttaccaaccatacagagtagtagtacttt
cttttgaacttctacatgcaccagcaactgtttgtggacctaaaaagtctactaatttggttaaaaacaa
atgtgtcaatttcaacttcaatggtttaacaggcacaggtgttcttactgagtctaacaaaaagtttctg
cctttccaacaatttggcagagacattgctgacactactgatgctgtccgtgatccacagacacttgaga
ttcttgacattacaccatgttcttttggtggtgtcagtgttataacaccaggaacaaatacttctaacca
ggttgctgttctttatcaggatgttaactgcacagaagtccctgttgctattcatgcagatcaacttact
cctacttggcgtgtttattctacaggttctaatgtttttcaaacacgtgcaggctgtttaataggggctg
aacatgtcaacaactcatatgagtgtgacatacccattggtgcaggtatatgcgctagttatcagactca
gactaattctcctcggcgggcacgtagtgtagctagtcaatccatcattgcctacactatgtcacttggt
gcagaaaattcagttgcttactctaataactctattgccatacccacaaattttactattagtgttacca
cagaaattctaccagtgtctatgaccaagacatcagtagattgtacaatgtacatttgtggtgattcaac
tgaatgcagcaatcttttgttgcaatatggcagtttttgtacacaattaaaccgtgctttaactggaata
gctgttgaacaagacaaaaacacccaagaagtttttgcacaagtcaaacaaatttacaaaacaccaccaa
ttaaagattttggtggttttaatttttcacaaatattaccagatccatcaaaaccaagcaagaggtcatt
tattgaagatctacttttcaacaaagtgacacttgcagatgctggcttcatcaaacaatatggtgattgc
cttggtgatattgctgctagagacctcatttgtgcacaaaagtttaacggccttactgttttgccacctt
tgctcacagatgaaatgattgctcaatacacttctgcactgttagcgggtacaatcacttctggttggac
ctttggtgcaggtgctgcattacaaataccatttgctatgcaaatggcttataggtttaatggtattgga
gttacacagaatgttctctatgagaaccaaaaattgattgccaaccaatttaatagtgctattggcaaaa
ttcaagactcactttcttccacagcaagtgcacttggaaaacttcaagatgtggtcaaccaaaatgcaca
agctttaaacacgcttgttaaacaacttagctccaattttggtgcaatttcaagtgttttaaatgatatc
ctttcacgtcttgacaaagttgaggctgaagtgcaaattgataggttgatcacaggcagacttcaaagtt
tgcagacatatgtgactcaacaattaattagagctgcagaaatcagagcttctgctaatcttgctgctac
taaaatgtcagagtgtgtacttggacaatcaaaaagagttgatttttgtggaaagggctatcatcttatg
tccttccctcagtcagcacctcatggtgtagtcttcttgcatgtgacttatgtccctgcacaagaaaaga
acttcacaactgctcctgccatttgtcatgatggaaaagcacactttcctcgtgaaggtgtctttgtttc
aaatggcacacactggtttgtaacacaaaggaatttttatgaaccacaaatcattactacagacaacaca
tttgtgtctggtaactgtgatgttgtaataggaattgtcaacaacacagtttatgatcctttgcaacctg
aattagactcattcaaggaggagttagataaatattttaagaatcatacatcaccagatgttgatttagg
tgacatctctggcattaatgcttcagttgtaaacattcaaaaagaaattgaccgcctcaatgaggttgcc
aagaatttaaatgaatctctcatcgatctccaagaacttggaaagtatgagcagtatataaaatggccat
ggtacatttggctaggttttatagctggcttgattgccatagtaatggtgacaattatgctttgctgtat
gaccagttgctgtagttgtctcaagggctgttgttcttgtggatcctgctgcaaatttgatgaagacgac
tctgagccagtgctcaaaggagtcaaattacattacacataa
MT039887_Wild-type_Spike_translated;
SEQ ID NO: 2
MFVFLVLLPLVSSQCVNLTTRTQLPPAYTNSFTRGVYYPDKVFRSSVLHSTQDLFLPFFS
NVTWFHAIHVSGTNGTKRFDNPVLPFNDGVYFASTEKSNIIRGWIFGTTLDSKTQSLLIV
NNATNVVIKVCEFQFCNDPFLGVYYHKNNKSWMESEFRVYSSANNCTFEYVSQPFLMDLE
LLALHRSYLTPGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASVYAWNRKRISN
CVADYSVLYNSASFSTFKCYGVSPTKLNDLCFTNVYADSFVIRGDEVRQIAPGQTGKIAD
YNYKLPDDFTGCVIAWNSNNLDSKVGGNYNYLYRLFRKSNLKPFERDISTEIYQAGSTPC
NGVEGFNCYFPLQSYGFQPTNGVGYQPYRVVVLSFELLHAPATVCGPKKSTNLVKNKCVN
FNFNGLTGTGVLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCLIGAEHVNNSY
ECDIPIGAGICASYQTQTNSPRRARSVASQSIIAYTMSLGAENSVAYSNNSIAIPTNFTI
SVTTEILPVSMTKTSVDCTMYICGDSTECSNLLLQYGSFCTQLNRALTGIAVEQDKNTQE
VFAQVKQIYKTPPIKDFGGFNFSQILPDPSKPSKRSFIEDLLFNKVTLADAGFIKQYGDC
LGDIAARDLICAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQDVVNQNAQALN
TLVKQLSSNFGAISSVLNDILSRLDKVEAEVQIDRLITGRLQSLQTYVTQQLIRAAEIRA
SANLAATKMSECVLGQSKRVDFCGKGYHLMSFPQSAPHGVVFLHVTYVPAQEKNFTTAPA
ICHDGKAHFPREGVFVSNGTHWFVTQRNFYEPQIITTDNTFVSGNCDVVIGIVNNTVYDP
LQPELDSFKEELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSCGSCCKFDEDD
SEPVLKGVKLHYT
SARS-CoV-2_Spike_codon-optimized; SEQ ID NO: 3:
atgtttgtctttcttgtgcttcttcctttggtcagttcacagtgtgtcaatcttactaccaggacacaac
ttccaccggcatacactaattctttcacacggggagtctattaccctgacaaagtgttcagaagttctgt
gctgcattcaacacaagatcttttcctcccgttctttagtaatgtgacctggtttcatgcaattcatgtg
tcaggaaccaatggtacaaagcggttcgataaccctgtgttgccgttcaatgacggagtctactttgctt
caactgagaaaagtaacatcattcggggatggattttcggaactacactcgattctaagacccagagtct
tttgattgtgaataacgcaacaaatgtcgtcatcaaagtctgtgagtttcaattttgtaatgatccattt
cttggagtgtattaccacaaaaacaataagtcatggatggagtcggagtttcgggtctattcttccgcta
ataattgcacttttgagtacgtctcacagccatttcttatggaccttgagggaaagcagggaaatttcaa
aaatcttagggagtttgtgtttaagaacattgacggatacttcaaaatctattcaaagcacactccgatc
aacctggtcagggatcttccgcaaggtttttcagcacttgaacctcttgtcgatctgcctattgggatca
acatcacaagattccaaactttgcttgctttgcatcggtcctatctcactccgggagattctagttcagg
gtggacagctggagcagctgcttactatgtgggttatcttcaacctaggacttttctgctgaaatacaat
gagaatggaaccatcactgacgctgtcgattgtgcactcgaccctctgtcagaaaccaaatgtactctca
aatctttcactgtggagaagggaatctaccaaacttcaaacttcagagtgcagcctacagagtctattgt
gcggtttccgaatatcactaacctttgcccgtttggagaagtgttcaatgccactagatttgcttcagtc
tatgcttggaaccggaaacggattagcaactgtgtcgctgactattcagtgctttacaattcagcatctt
tctcaacctttaagtgctacggagtgtctccgacaaaactcaatgacctttgtttcactaatgtctatgc
tgactcgttcgtcattcggggagatgaagtccggcagattgccccagggcaaacaggcaaaatcgctgac
tataactacaaactgcctgacgactttaccggatgcgtcattgcatggaatagtaacaatctcgattcta
aagtcggtggaaactacaattacctctatagactttttcggaagtcaaatctcaaaccttttgaacggga
catctctactgagatctaccaagctggtagtacaccatgtaatggagtggagggattcaattgctacttc
cctttgcagtcctacggtttccaacccacaaatggagtcggttaccagccttatagggtcgtcgtcttgt
cgtttgagttgcttcatgcacctgctacagtctgtggtccaaagaaatctactaatcttgtcaaaaacaa
atgtgtgaatttcaacttcaatggacttactggtactggtgtccttactgaatctaacaaaaagtttctg
ccttttcaacaatttggtagagatattgctgatactactgatgctgtccgggacccacagactcttgaaa
ttctcgacattactccatgttcgtttggaggtgtctcagtcattacacctggtactaatactagcaacca
ggtcgctgtcttgtatcaggatgtgaactgcacagaagtgccagtggctatccatgcagatcaattgact
ccaacttggcgggtctattcgactggatcaaatgtgtttcaaactagggccggatgtttgattggagctg
agcatgtcaataactcttatgagtgtgacattccgattggagcaggaatctgcgcttcttatcaaactca
aacaaattcaccaagaagagcacggagcgtcgcttcacaatctatcatcgcatacacaatgtcacttgga
gctgagaatagtgtcgcttactctaacaattcaattgccattcctactaattttacaatctcagtcacca
ccgaaatccttccagtgtcaatgaccaagacttcagtcgattgtacaatgtacatctgtggtgattctac
cgagtgtagtaatcttctccttcaatatgggtcattttgtacacagctcaatagagctttgactggaatc
gcagtggagcaagacaaaaacactcaagaggtctttgcacaagtcaagcaaatctacaaaacaccaccga
tcaaagattttggaggattcaatttctcacaaattcttccggacccgtcaaaacctagcaagagatcttt
catcgaagatttgcttttcaataaggtgacacttgctgatgctggtttcatcaaacaatatggcgattgt
cttggtgatatcgcagctcgggacctgatctgtgcacaaaagtttaacggactgactgtcctccccccac
ttctgactgatgagatgattgctcaatacacaagtgctctgcttgctggaactatcacatcgggatggac
ctttggagctggagctgcacttcaaattccttttgctatgcaaatggcttatcggtttaacggtattggt
gtcacacagaatgtcctttatgaaaatcaaaagcttatcgccaaccaattcaatagtgctattgggaaga
ttcaagacagtctttcatctactgcttctgctctcggtaaacttcaagatgtcgtcaatcaaaatgctca
agcattgaacactcttgtcaagcagcttagtagtaactttggagctatttcttctgtgcttaatgacatc
ctttcacggcttgacaaggtcgaagcagaggtgcaaattgataggcttatcactgggagacttcaaagtc
ttcagacctatgtcactcaacaacttattcgggctgctgaaatccgggcatcagccaatttggcagccac
taagatgtcagagtgtgtcttgggacaatctaaaagagtcgatttctgtggtaaggggtatcatcttatg
tcattcccgcagtcagctccgcatggagtcgtctttttgcatgtgacttatgtcccagcacaagaaaaga
acttcactactgcaccagctatttgtcatgatggaaaagctcactttccacgggaaggagtgtttgtgtc
taatggaactcattggttcgtcacacagaggaatttctatgaaccgcagattatcactacagacaacact
tttgtcagtggtaactgcgatgtcgtcattgggattgtcaacaacaccgtgtacgatccgcttcaaccgg
aactcgattctttcaaggaggaacttgacaaatatttcaagaatcatacatcacctgatgtggatcttgg
agacatctcgggaatcaatgcatcggtcgtcaatatccaaaaggaaattgaccggttgaatgaggtggcc
aagaatttgaatgagtcacttattgatctccaagagttgggaaagtatgaacagtatatcaaatggccat
ggtacatttggcttggattcatcgctggcctgattgccatcgtcatggtgaccattatgctctgttgtat
gacctcatgctgttcttgtctcaagggatgttgttcatgtgggtcgtgctgcaaatttgatgaggacgac
tcagaaccagtgctcaaaggagtcaaactccattacacttga
SARS-CoV-2_Spike_codon-optimized_RNA
auguuugucuuucuugugcuucuuccuuuggucaguucacagugugucaaucuuacuaccaggacacaac
uuccaccggcauacacuaauucuuucacacggggagucuauuacccugacaaaguguucagaaguucugu
gcugcauucaacacaagaucuuuuccucccguucuuuaguaaugugaccugguuucaugcaauucaugug
ucaggaaccaaugguacaaagcgguucgauaacccuguguugccguucaaugacggagucuacuuugcuu
caacugagaaaaguaacaucauucggggauggauuuucggaacuacacucgauucuaagacccagagucu
uuugauugugaauaacgcaacaaaugucgucaucaaagucugugaguuucaauuuuguaaugauccauuu
cuuggaguguauuaccacaaaaacaauaagucauggauggagucggaguuucgggucuauucuuccgcua
auaauugcacuuuugaguacgucucacagccauuucuuauggaccuugagggaaagcagggaaauuucaa
aaaucuuagggaguuuguguuuaagaacauugacggauacuucaaaaucuauucaaagcacacuccgauc
aaccuggucagggaucuuccgcaagguuuuucagcacuugaaccucuugucgaucugccuauugggauca
acaucacaagauuccaaacuuugcuugcuuugcaucgguccuaucucacuccgggagauucuaguucagg
guggacagcuggagcagcugcuuacuauguggguuaucuucaaccuaggacuuuucugcugaaauacaau
gagaauggaaccaucacugacgcugucgauugugcacucgacccucugucagaaaccaaauguacucuca
aaucuuucacuguggagaagggaaucuaccaaacuucaaacuucagagugcagccuacagagucuauugu
gcgguuuccgaauaucacuaaccuuugcccguuuggagaaguguucaaugccacuagauuugcuucaguc
uaugcuuggaaccggaaacggauuagcaacugugucgcugacuauucagugcuuuacaauucagcaucuu
ucucaaccuuuaagugcuacggagugucuccgacaaaacucaaugaccuuuguuucacuaaugucuaugc
ugacucguucgucauucggggagaugaaguccggcagauugccccagggcaaacaggcaaaaucgcugac
uauaacuacaaacugccugacgacuuuaccggaugcgucauugcauggaauaguaacaaucucgauucua
aagucgguggaaacuacaauuaccucuauagacuuuuucggaagucaaaucucaaaccuuuugaacggga
caucucuacugagaucuaccaagcugguaguacaccauguaauggaguggagggauucaauugcuacuuc
ccuuugcaguccuacgguuuccaacccacaaauggagucgguuaccagccuuauagggucgucgucuugu
cguuugaguugcuucaugcaccugcuacagucugugguccaaagaaaucuacuaaucuugucaaaaacaa
augugugaauuucaacuucaauggacuuacugguacugguguccuuacugaaucuaacaaaaaguuucug
ccuuuucaacaauuugguagagauauugcugauacuacugaugcuguccgggacccacagacucuugaaa
uucucgacauuacuccauguucguuuggaggugucucagucauuacaccugguacuaauacuagcaacca
ggucgcugucuuguaucaggaugugaacugcacagaagugccaguggcuauccaugcagaucaauugacu
ccaacuuggcgggucuauucgacuggaucaaauguguuucaaacuagggccggauguuugauuggagcug
agcaugucaauaacucuuaugagugugacauuccgauuggagcaggaaucugcgcuucuuaucaaacuca
aacaaauucaccaagaagagcacggagcgucgcuucacaaucuaucaucgcauacacaaugucacuugga
gcugagaauagugucgcuuacucuaacaauucaauugccauuccuacuaauuuuacaaucucagucacca
ccgaaauccuuccagugucaaugaccaagacuucagucgauuguacaauguacaucuguggugauucuac
cgaguguaguaaucuucuccuucaauaugggucauuuuguacacagcucaauagagcuuugacuggaauc
gcaguggagcaagacaaaaacacucaagaggucuuugcacaagucaagcaaaucuacaaaacaccaccga
ucaaagauuuuggaggauucaauuucucacaaauucuuccggacccgucaaaaccuagcaagagaucuuu
caucgaagauuugcuuuucaauaaggugacacuugcugaugcugguuucaucaaacaauauggcgauugu
cuuggugauaucgcagcucgggaccugaucugugcacaaaaguuuaacggacugacuguccuccccccac
uucugacugaugagaugauugcucaauacacaagugcucugcuugcuggaacuaucacaucgggauggac
cuuuggagcuggagcugcacuucaaauuccuuuugcuaugcaaauggcuuaucgguuuaacgguauuggu
gucacacagaauguccuuuaugaaaaucaaaagcuuaucgccaaccaauucaauagugcuauugggaaga
uucaagacagucuuucaucuacugcuucugcucucgguaaacuucaagaugucgucaaucaaaaugcuca
agcauugaacacucuugucaagcagcuuaguaguaacuuuggagcuauuucuucugugcuuaaugacauc
cuuucacggcuugacaaggucgaagcagaggugcaaauugauaggcuuaucacugggagacuucaaaguc
uucagaccuaugucacucaacaacuuauucgggcugcugaaauccgggcaucagccaauuuggcagccac
uaagaugucagagugugucuugggacaaucuaaaagagucgauuucugugguaagggguaucaucuuaug
ucauucccgcagucagcuccgcauggagucgucuuuuugcaugugacuuaugucccagcacaagaaaaga
acuucacuacugcaccagcuauuugucaugauggaaaagcucacuuuccacgggaaggaguguuuguguc
uaauggaacucauugguucgucacacagaggaauuucuaugaaccgcagauuaucacuacagacaacacu
uuugucagugguaacugcgaugucgucauugggauugucaacaacaccguguacgauccgcuucaaccgg
aacucgauucuuucaaggaggaacuugacaaauauuucaagaaucauacaucaccugauguggaucuugg
agacaucucgggaaucaaugcaucggucgucaauauccaaaaggaaauugaccgguugaaugagguggcc
aagaauuugaaugagucacuuauugaucuccaagaguugggaaaguaugaacaguauaucaaauggccau
gguacauuuggcuuggauucaucgcuggccugauugccaucgucauggugaccauuaugcucuguuguau
gaccucaugcuguucuugucucaagggauguuguucaugugggucgugcugcaaauuugaugaggacgac
ucagaaccagugcucaaaggagucaaacuccauuacacuuga

### EXAMPLES

### Example 1

### Incorporation of the Coding Sequences for SARS-CoV-2 Spike Protein into the VEE Alphavirus RNA Replicon Particles

### Introduction

RNA viruses can be used as vector-vehicles for introducing vaccine antigens that have been genetically engineered into their genomes. However, their use to date has been limited primarily to incorporating viral antigens into the RNA virus and then introducing the virus into a recipient host. The result is the induction of protective antibodies against the incorporated viral antigens. Alphavirus RNA replicon particles have been used to encode pathogenic antigens. Such alphavirus replicon platforms have been developed from several different alphaviruses, including Venezuelan equine encephalitis virus (VEE) [Pushko et al., Virology 239:389-401 (1997)], Sindbis (SIN) [Bredenbeek et al., Journal of Virology 67:6439-6446 (1993) the contents of which are hereby incorporated herein in their entireties], and Semliki Forest virus (SFV) [Liljestrom and Garoff, Biotechnology (NY) 9:1356-1361 (1991), the contents of which are hereby incorporated herein in their entireties]. Moreover, alphavirus RNA replicon particles are the basis for several USDA-licensed vaccines for swine and poultry. These include: Porcine Epidemic Diarrhea Vaccine, RNA Particle (Product Code 19U5.P1), Swine Influenza Vaccine, RNA (Product Code 19A5.D0), Avian Influenza Vaccine, RNA (Product Code 1905.D0), and Prescription Product, RNA Particle (Product Code 9PP0.00).

### Alphavirus RNA Replicon Construction

A vaccine is prepared comprising an alphavirus RNA replicon particle encoding codon optimized SARS-CoV-2 Spike Protein.

### Generation of SARS-CoV-2 Spike Protein Gene Replicon Particles (RPs)

The VEE replicon vector for use to express the SARS-CoV-2 Spike gene is constructed as previously described [see, U.S. Pat. No. 9,441,247 B2; the contents of which are hereby incorporated herein by reference], with the following modifications. The TC-83-derived replicon vector “pVEK” [disclosed and described in U.S. Pat. No. 9,441,247 B2] is digested with restriction enzymes AscI and PacI to create the vector “pVHV”. The spike protein gene sequence from SARS-CoV-2, strain 2019-nCoV/U.S.A.-WI1/2020 (GenBank accession MT039887), was codon-optimized and is synthesized with flanking AscI and PacI sites. The synthetic gene and pVHV vector are each digested with AscI and PacI enzymes and ligated to create vector “pVHV-SARS-CoV-2-Spike”. Plasmid batches are sequenced to confirm the correct vector and insert identities.

Production of TC-83 RNA replicon particles (RP) is conducted according to methods previously described [U.S. Pat. No. 9,441,247 B2 and U.S. Pat. No. 8,460,913 B2; the contents of which are hereby incorporated herein by reference]. Briefly, pVHV-SARS-CoV-2-Spike replicon vector DNA and helper DNA plasmids are linearized with NotI restriction enzyme prior to in vitro transcription using MegaScript T7 RNA polymerase and cap analog. Importantly, the helper RNAs that are used in the production lack the VEE subgenomic promoter sequence, as previously described [Kamrud et al., J Gen Virol. 91(Pt 7):1723-1727 (2010)]. Purified RNA for the replicon and helper components are combined and mixed with a suspension of Vero cells, electroporated in 4 mm cuvettes, and returned to serum-free culture media. Following overnight incubation, alphavirus RNA replicon particles are purified from the cells and media by passing the suspension through a depth filter, washing with phosphate buffered saline containing 5% sucrose (w/v), and finally eluting the retained RP with 400 mM NaCl+5% sucrose (w/v) buffer. Eluted RP are passed through a 0.22 micron membrane filter, and dispensed into aliquots for storage. Titer of functional RP is determined by immunofluorescence assay on infected Vero cell monolayers. The resulting propagation-defective alphavirus RNA replicon particle encoding codon optimized SARS-CoV-2 spike protein can then be placed into a non-adjuvanted or adjuvanted vaccine formulation and administered to a feline or a ferret.

### Example 2

### Humoral and Cellular Immune Responses Induced by SARS-COV-2 Spike Antigens Using VEEV RP Vaccines in Guinea Pigs

### Materials and Methods

### Animals and husbandry

Female SPF guinea pigs (Dunkin Hartley) were obtained from Envigo at a minimum weight of 350 grams, randomly allocated to experimental groups, and individually marked using color coded tags. Baseline clinical observations were documented throughout the study period.

Baseline clinical observations including body temperatures were documented throughout the study period.

### Generation of SARS-CoV-2 Spike Gene RP Vaccines

The Spike_wt gene sequence from SARS-CoV-2, strain 2019-nCoV/U.S.A.-WI1/2020 (GenBank accession MT039887) was codon-optimized and inserted in the pVHV vector.

Production of TC-83 RNA RPs was conducted by the methods described above.

### Guinea Pig Study

SPF guinea pigs with a minimum weight of 350 grams were randomly divided over the non-vaccinated control group, and RP-Spike-wt vaccinated group (n=6 per group). One week after placement, animals remained either non-vaccinated or received a prime vaccination of 1×10E7 RP dose intramuscularly (0.1 ml in each leg muscle). Three weeks after prime vaccination the animals received a booster vaccination of 1×10E7 RP dose intramuscular (0.1 ml in each leg muscle). Six weeks after the booster the vaccination animals received a second booster vaccination and 7 days later animals were sacrificed. Terminal blood was taken for lymphocyte stimulation tests (LST). At the day of the booster vaccination, and at 2-week intervals until 6 weeks after boost vaccination, clotted blood was taken using cardiac-puncture and the serum was used to determine systemic antibody titers.

### Surrogate Virus Neutralization Assay Guinea Pig SERA

The SARS-CoV-2 Surrogate Virus Neutralization Test Kit from GenScript was used according to the manufacturer's instructions. Briefly, sera were diluted in sample dilution buffer, mixed 1:1 with HRP-RBD, and incubated for 30 minutes at 37° C. Next, samples were put in a 96-well plate containing ACE2 receptor coated on the surface and incubated 15 minutes at 37° C. Unbound HRP-RBD was washed away and remaining horse radish peroxide (HRP) was visualized using 3,3′,5,5′-tetramethylbenzidine (TMB) substrate and measured at OD450.

### ELISA for Estimating Spike Ectodomain Antibody Titers in Sera

Purified SARS-CoV-2 Spike ectodomain were diluted in Dulbecco's phosphate-buffered saline (DPBS) [without Ca and Mg, Lonza, 17-512F] and coated onto 96-well plates (MaxiSorp-ThermoFisher, or High binding-Greiner Bio-one) using 10 nM (10 pmols/mL), and incubated overnight at 4° C. The next morning the plates were washed three times with an ELISA plate washer (ImmunoWash 1575, BioRad) using 0.25 mL wash solution/well (DPBS, 0.05% Tween 20), then blocked with 250 μL blocking solution (5% milk-Protifar, Nutricia, 0.1% Tween 20 in DPBS) for 2 hours at RT (room temperature). Afterwards the blocking solution was discarded. Then four-fold serial dilutions of the sera (prepared in the blocking solution, in duplicates or triplicates) were added to the corresponding wells and incubated for 1 hour at Room Temperature (RT). Each plate contained positive control (guinea pig sera diluted to obtain an OD450 of ˜2) and negative control wells. The plates were washed again 3 times before being incubated with the HRP-containing antibody—Goat anti-Guinea pig (IgG-HRPO, Jackson Lab 106-035-003, 1:8000) for 1 hour at RT. The last wash steps were performed, followed by an incubation for 10 minutes at RT with 100 μL/well Super Sensitive TMB (Surmodics, TMBS-1000-01). Reactions were stopped by adding 100 μL/well of 12.5% H₂SO₄ (Millipore, 1.00716.1000). Absorbance at 450 nm was measured at 30 minutes with an ELx808 BioTek plate reader.

### T-Cell Stimulation Test (LST)

Blood was collected and lymphocytes were isolated using Sepmate tube (Stemcell) containing Histopaque 1083 according to manufacturer's instructions. Briefly, K3-EDTA blood was diluted 1:2 in RPMI-1640 medium and pelleted for 10 minutes at 1200×g. The cells in the top layer of the tubes were collected, placed in a clean tube containing RPMI-1640 and pelleted for 7 minutes at 400×g. The cells were washed once with RPMI-1640 medium and pelleted for 7 minutes at 400×g. Cell concentrations were counted and 1×10E7 cells were stained with carboxyfluorescein succinimidyl ester (CFSE) for 20 minutes at 37° C. The cells were washed once with RPMI-1640 and 5×10E5 cells from each animal were stimulated with either medium, ConA (10 μg/ml), or purified SARS-CoV-2 S1 antigen (5, 2.5, 1.25, 0.62, 0.31, or 0.15 μg/ml) in duplicate. Three days after stimulation, cell proliferation was measured using the FACS-Verse.

### Results

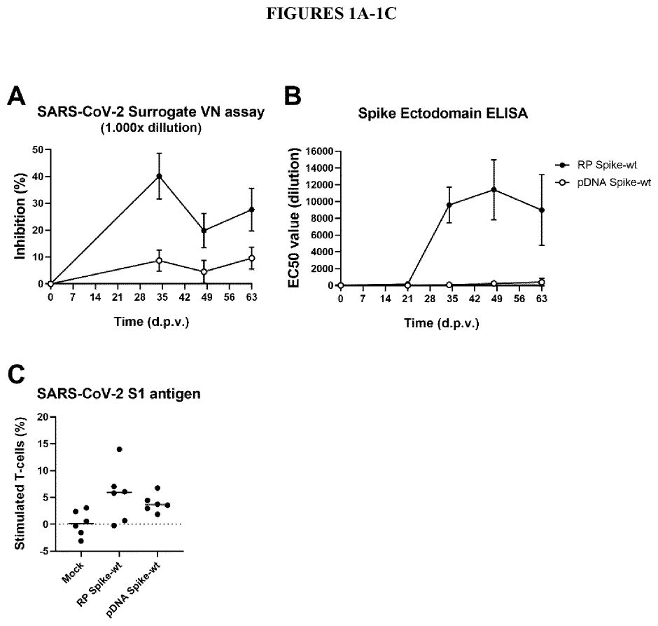

Immunogenicity of the Spike-wt antigen was assessed in a guinea pig model in which the VEEV RP vector vaccines were administered intramuscularly. After prime vaccination, all animals showed seroconversion as assessed by a commercially available SARS-CoV-2 surrogate VN test that measures antibody titers interfering with Spike-receptor binding. After boost vaccination of the guinea pigs with either the VEEV RP or plasmid DNA vector vaccines producing the SARS-CoV-2 Spike antigen, peak serum neutralizing antibody titers were detected using a SARS-CoV-2 surrogate VN assay at 34 days post vaccination (FIG. 1A). At 49 and 63 days post vaccination neutralizing serum antibody titers decreased slightly. In general, the VEEV RP vaccine induced superior serum antibody titers compared to the plasmid DNA vaccine. Similar results were observed when total antibody titers were determined using a SARS-CoV-2 Spike Ectodomain ELISA (FIG. 1B). In this assay, peak serum antibody titers were detected at 49 days post vaccination and again the VEEV RP vaccine induced superior serum antibody titers compared to the plasmid DNA vaccine.

The VEEV RP vector platform is known for its efficient induction of both humoral as well as cellular responses. To assess the level of cellular responses induced by the RP vaccine candidates, a third immunization was performed and seven days later lymphocytes were isolated for a lymphocyte stimulation test (LST). All isolated lymphocytes stimulated with ConA resulted in >80% proliferation titers. The VEEV Replicon Particle vaccine producing the SARS-CoV-2 Spike antigen was able to induce a strong cellular immune response in guinea pigs (FIG. 1C). The antibody responses induced by the VEEV RP vaccine induced superior titers compared to the plasmid DNA vaccine, while both vaccine platforms were able to induce comparable cellular immune responses.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

It is further to be understood that all base sizes or amino acid sizes, and all molecular weight or molecular mass values, given for nucleic acids or polypeptides are approximate, and are provided for description.

1. An immunogenic composition comprising a Venezuelan Equine Encephalitis (VEE) alphavirus RNA replicon particle that encodes a first SARS-CoV-2 protein antigen.

2. The immunogenic composition of claim 1, wherein the first SARS-CoV-2 protein antigen is a spike protein or an immunogenic fragment thereof.

3. The immunogenic composition of claim 2, wherein the VEE alphavirus RNA replicon particle further encodes a second antigen selected from the group consisting of a second SARS-CoV-2 spike protein that originates from a different strain of SARS-CoV-2 than the first SARS-CoV-2 spike protein originates from, a feline calicivirus (FCV) capsid protein, a feline leukemia virus (FeLV) envelope protein, and a rabies virus glycoprotein (G).

4. The immunogenic composition of claim 2, that comprises one or more additional VEE alphavirus RNA replicon particles which encodes a second SARS-CoV-2 spike protein that originates from a different strain of SARS- CoV-2 than the first SARS-CoV-2 spike protein originates from.

5. The immunogenic composition of claim 1, wherein the first SARS-CoV-2 spike protein comprises an amino acid sequence comprising at least 98% identity with the amino acid sequence of SEQ ID NO: 2.

6. The immunogenic composition of claim 1, wherein the first SARS-CoV-2 spike protein is encoded by a nucleotide sequence comprising at least 98% identity with the nucleotide sequence of SEQ ID NO: 4.

7. A vaccine to aid in reducing shedding of SARS-CoV-2 in a feline or a ferret due to an infection of SARS-CoV-2 in the feline or the ferret comprising the immunogenic composition of claim 1, and a pharmaceutically acceptable carrier.

8. The vaccine of claim 7, that further comprises at least one non-SARS-CoV-2 antigen for eliciting protective immunity to a non-SARS-CoV-2 pathogen.

9. The vaccine of claim 8, wherein the non-SARS-CoV-2 pathogen is an inactivated or an attenuated non-SARS-CoV-2 pathogen.

10. The vaccine of claim 7, that further comprises a VEE alphavirus RNA replicon particle comprising a nucleotide sequence encoding at least one antigen or immunogenic fragment thereof that originates from the non-SARS-CoV-2 pathogen.

11. The vaccine of claim 8, wherein the non-SARS-CoV-2 pathogen is selected from the group consisting of feline calicivirus (FCV), feline leukemia virus (FeLV), feline panleukopenia virus (FPLV), feline rhinotracheitis virus (FVR), Chlamydophila felis, rabies virus, canine influenza virus (CIV), canine parvovirus (CPV), canine distemper (CDV), and any combination thereof.

12. The vaccine of claim 11, wherein the non-SARS-CoV-2 pathogen is selected from the group consisting of FCV, FeLV, FPLV, FVR, Chlamydophila felis, rabies virus, canine influenza virus (CIV), and any combination thereof.

13. The vaccine of claim 11, wherein the non-SARS-CoV-2 pathogen is selected from the group consisting of canine parvovirus (CPV), canine distemper (CDV), and the combination thereof.

14. The vaccine of claim 7, that comprises an adjuvant.

15. The vaccine of claim 7, that is a non-adjuvanted vaccine.

16. A method of immunizing a mammal against SARS-CoV-2 comprising administering to the mammal an immunologically effective amount of the vaccine of claim 7; wherein the mammal is selected from the group of a feline and a ferret.

17. The method of claim 16, wherein the mammal is a feline.

18. The method of claim 17, wherein the feline is a domestic cat.

19. The method of claim 16, wherein the mammal is a ferret.
